(19) United States
(12) Patent Application Publication
(10) Pub. No.: US 2025/0336513 A1
(43) Pub. Date: Oct. 30, 2025

(54) SYSTEM AND METHOD FOR LOW-MEMORY PARSING OF ELECTROPHYSIOLOGICAL SIGNALS FROM NEURONS TO IDENTIFY NEURON ACTIVITY

(71) Applicant: THE GOVERNING COUNCIL OF THE UNVERSITY OF TORONTO, Toronto (CA)

(72) Inventors: Eugene Ching Hong SHA, Toronto (CA); Ameer ABD ELHADI, Stoney Creek (CA); Andy Wei LIU, Mississauga (CA); Andreas MOSHOVOS, North York (CA)

(21) Appl. No.: 19/188,299

(22) Filed: Apr. 24, 2025

Related U.S. Application Data
(60) Provisional application No. 63/638,159, filed on Apr. 24, 2024.

(51) Int. Cl. G16H 40/63; A61B 5/00; A61B 5/369

(52) U.S. Cl. CPC G16H 40/63; A61B 5/369; A61B 5/7246

(57) ABSTRACT

There is provided a system and method for low-memory parsing of electrophysiological signals from neurons to identify neuron activity. The method including: receiving electrophysiological signals from neural probes; digitizing the received electrophysiological signals and serializing the digitized signals across a plurality of channels; performing filtering on the digitized electrophysiological signals of each channel; performing whitening over the filtered samples of a group of associated channels; detecting whether the whitened samples for each channel includes a spike, the samples include the spike where a centered peak exceeds a threshold and is greater in value than a predetermined number of neighboring samples; determining a matching neuron for the detected spike as an identification of neuron activity; and outputting the identification of neuron activity.

### TECHNICAL FIELD

The following relates, generally, to brain signal interpretation; and more particularly, to a system and method for low-memory parsing of electrophysiological signals from neurons to identify neuron activity.

### BACKGROUND

Parsing of electrophysiological signals from neurons recorded from a patient's brain to identify if, when, and which particular neurons fire is commonly referred to as ‘spike sorting’. Spike sorting is a particularly difficult computational task in neuroscience due to a substantially growing scale of recording technologies and complexity in traditional spike sorting algorithms.

Broadly speaking, the human brain comprises billions of neurons that communicate through electrophysiological signals called spikes, which serve as the fundamental units of brain communication. To better understand complex brain behaviors and structures, neuroscientists employ spike sorting, which attributes spikes to their respective firing neurons. This single-neuron activity reveals higher-order brain functionality. Real-time interaction via neuronal communication enables substantial advances, e.g., motor control for paralysis patients, epilepsy detection and mitigation, treatment of Parkinson's disease, and cognitive control. Apart from some limited applications, larger scale applications remain unrealized due to the many impediments to perform spike sorting at a high-scale. Various aspects need to scale to more than tens of thousands of neurons for such larger-scale applications to begin to materialize: 1) implantable voltage sensors, 2) an analog-to-digital front-end voltage converter, and 3) a digital processing back-end. It is possible to reach larger-scales with the implantable probes and analog-to-digital conversion aspects. However, larger-scale brain-machine applications generally hinge upon the digital back-end to observe and act upon activity across orders of magnitude more neurons, and to do so in real-time using wearable, energy-efficient systems that operate autonomously for long periods of time.

### SUMMARY

In an aspect of the present invention, there is provided a method for low-memory parsing of electrophysiological signals from neurons to identify neuron activity, the method comprising: receiving electrophysiological signals from neural probes; digitizing the received electrophysiological signals and serializing the digitized signals across a plurality of channels; performing filtering on the digitized electrophysiological signals of each channel; performing whitening over the filtered samples of a group of associated channels; detecting whether the whitened samples for each channel comprises a spike, the samples comprise the spike where a centered peak exceeds a threshold and is greater in value than a predetermined number of neighboring samples; determining a matching neuron for the detected spike as an identification of neuron activity; and outputting the identification of neuron activity.

In a particular case of the method, filtering is performed using a third order Butterworth infinite impulse response bandpass filter with a cascaded biquads.

In another case of the method, performing filtering comprises performing time-domain multiplexing with the digitized electrophysiological signals of multiple channels.

In yet another case of the method, the group of associated channels are arranged in a uniform grid for whitening.

In yet another case of the method, performing whitening comprises determining, for each one of the group of associated channels, a dot product of neighboring samples of the channel and a predetermined whitening matrix.

In yet another case of the method, detecting whether the whitened samples for each channel comprises a spike comprises determining a central channel of the group of associated channels that has the spike.

In yet another case of the method, determining the matching neuron comprises determining a dot product of the neighboring samples with one or more templates, the matching neuron corresponding to a highest magnitude dot product.

In yet another case of the method, templates for template matching are each stored as a fixed portion and a variable portion which is decompressible.

In yet another case of the method, performing template matching comprises decompressing the variable portion of each of the templates, wherein decompressing the variable portion of each template comprises overriding a decompressed value with an outlier.

In yet another case of the method, determining the matching neuron comprises using a trained machine learning model to determine the matching neuron for identification of neuron activity.

In another aspect, there is provided a controller for low-memory parsing of electrophysiological signals from neurons to identify neuron activity, the controller comprising hardware to receive instructions from one or more memory units to execute: an input module to receive electrophysiological signals from one or more neural probes that capture the electrophysiological signals, to digitize the received electrophysiological signals, and to serialize 8 the digitized signals across a plurality of channels; a filtering module to perform filtering on the digitized electrophysiological signals of each channel; a whitening module to perform whitening over the filtered samples of a group of associated channels; a detection module to detect whether the whitened samples for each channel comprises a spike, the samples comprise the spike where a centered peak exceeds a threshold and is greater in value than a predetermined number of neighboring samples; a matching module to determine a matching neuron for the detected spike as an identification of neuron activity; and an output module to output the identification of neuron activity.

In a particular case of the controller, the whitening module comprises a neighborhood buffer to receive the filtered samples of the group of associated channels, the neighborhood buffer comprising a transpose buffer that feeds a neighborhood staging.

In another case of the controller, the detection module comprises a sample buffer to receive a last number of samples per channel, and a spike aging counter to perform peak detection for the predetermined number of neighboring samples.

In yet another case of the controller, filtering is performed by the filtering module using a third order Butterworth infinite impulse response bandpass filter with a cascaded biquads.

In yet another case of the controller, filtering is performed by the filtering module by performing time-domain multiplexing with the digitized electrophysiological signals of multiple channels.

In yet another case of the controller, the group of associated channels are arranged in a uniform grid for whitening.

In yet another case of the controller, the whitening module performs whitening by determining, for each one of the group of associated channels, a dot product of neighboring samples of the channel and a predetermined whitening matrix.

In yet another case of the controller, the detection module detects whether the whitened samples for each channel comprise a spike by determining a central channel of the group of associated channels that has the spike.

In yet another case of the controller, the matching module determines the matching neuron by determining a dot product of the neighboring samples with one or more templates or comprises using a trained machine learning model to determine the matching neuron for identification of neuron activity.

In another aspect, there is provided a system for low-memory parsing of electrophysiological signals from neurons to identify neuron activity, the system comprising the controller, a power source connected to the controller, and the one or more neural probes electrically connected to the controller.

These and other aspects are contemplated and described herein. It will be appreciated that the foregoing summary sets out representative aspects of the system and method to assist skilled readers in understanding the following detailed description.

### DESCRIPTION OF THE DRAWINGS

A greater understanding of the embodiments will be had with reference to the Figures, in which:

FIG. 1 is a diagram showing an example brain-machine interface pipeline;

FIG. 2 is a diagram showing various stages of an online spike sorter, illustrating functional stages and the associated input and output data;

FIG. 3A is a chart illustrating compute costs, and FIG. 3B is a chart illustrating memory costs, for an online spike sorting pipeline;

FIG. 4A is a chart illustrating waveforms in a K-means clusters and FIG. 4B is a chart showing an evaluation sweeping configurations of codebook sizes and outlier thresholds for K-means clustering and indirect quantization against their accuracies on SpikeForest datasets;

FIG. 5 shows memory cost of an online spike sorter for 10 K channels and 20 Hz firing rate, with the leftmost chart showing absolute difference and the rightmost chart showing normalized memory costs;

FIG. 6 shows a conceptual block diagram of a system for low-memory parsing of electrophysiological signals from neurons to identify neuron activity, according to an embodiment;

FIG. 7 is a diagram showing an example of a spike sorting pipeline in accordance with the system of FIG. 6;

FIGS. 8A and 8B are diagrams showing an example of a neighborhood transpose buffer in accordance with the system of FIG. 6;

FIGS. 9A to 9D are diagrams showing spike sorting stages, where FIG. 9A shows a sample buffering stage, FIG. 9B shows a peak detection stage, FIG. 9C shows a spike aging stage, and FIG. 9D shows a neighbourhood peak stage;

FIG. 10 is a diagram showing a schedule of data, where incoming samples from each channel are to be digitized and serialize according to such schedule and outputs of filtering and whitening also follow such schedule; and

FIG. 11 is a flowchart of a method for low-memory parsing of electrophysiological signals from neurons to identify neuron activity, according to an embodiment.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

Any module, unit, component, server, computer, terminal or device exemplified herein that executes instructions may include or otherwise have access to computer readable media such as 8 storage media, computer storage media, or data storage devices (removable and/or non-removable). Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Near-brain implants, including neural prosthetics and brain-machine interfaces (BMIs), generally require a well-defined power budget due to two primary considerations. Firstly, safety is a crucial concern for many near-brain implants. Excessive power consumption can lead to heat generation, potentially damaging sensitive brain tissue. Therefore, a power budget helps ensure that the implant operates within safe temperature limits, safeguarding the well-being of the patient.

Battery life is another paramount concern. These devices often rely on batteries for power, and optimizing power usage is essential to prolong battery life. Longer battery life reduces the frequency of recharging batteries, enhancing the patient's quality of life and reducing associated risks. The power budget of near-brain implants can vary significantly depending on the specific device, its intended application, and technological advancements. However, recent findings and clinical considerations strongly suggest that a power budget of 2 W should be considered optimal.

Generally, most processing systems, e.g., CPUs and GPUs, are far from capable of meeting the stringent combination of processing capability and power efficiency needed to keep up with these advances. Currently, only low-scale systems are able to meet such demands. For example, such approaches with only a few hundreds of neurons require tethering to a server and offline analysis; which severely limits their utility.

Fully-implantable devices illustrate the inherent challenges in device design due to their stringent constraints. In addition to the portability of a near-brain implant, fully-implantable devices are generally restricted to smaller form factors, have stringent durability and longevity considerations, and must adhere to stricter power budgets due to the 1° C. thermal safety threshold determined by the International Organization for Standardization to prevent brain damage and cell death. The power budget is limited to 47-81 mW, but this can reduce further depending on the device's spatial footprint. Various current approaches scale to only hundreds of channels within a 50 mW power budget. Additionally, current neural amplifiers consume approximately 0.5-10 AW per channel. For ten thousand channels, amplifiers alone can consume 5-100 mW, surpassing the entire power budget

Advantageously, the present embodiments address the above limitations in the art by providing a low-power architecture to scale up input neuron count in the thousands. Advantageously, the present embodiments allow for spike sorting in real-time for thousands of probe channels and in a wearable form factor with a hardware pipeline that enables spike sorting at the scale of tens of thousands of neurons.

As described herein, software-based template matching for spike sorting cannot scale up to thousands of neurons due to memory and computation needs far exceeding even high-end processing cores. Indicatively, keeping pace with the input stream from 10 K channels requires greater than 100 B instructions/second, out of which 75 B is solely to identify windows of interest where spikes may be occurring. This is challenging even for high-end CPUs and GPUs, let alone for a wearable, energy efficient systems. Memory demands are also problematic for scaling as they reach 16 M elements for template storage alone.

In order to overcome the above limitations, the present inventors developed a hardware architecture for high-channel count spike sorting; which example experiments illustrate can be used for up to 10 K channels or 30 K neurons in wearable applications. The architecture includes at least two major components. Firstly, a series of fixed-logic processing stages which aim to denoise input waveforms and to identify areas of interest. These are spatiotemporal windows into channel streams which may contain a spike. Each window is centered around a local peak in the input signals and contains samples around the peak from all relevant neighboring channels. Secondly, a template matching component, where the window is compared against prerecorded templates in order to identify the source neuron. The hardware architecture can use a flexible vector processing unit to perform the template matching. Advantageously, the hardware architecture provides a lightweight template compression approach that makes it practical to store the templates. Advantageously, the front-end, “window of interest” unit can be used with other back-end spike identification approaches. For example, a machine-learning back-end is provided herein that uses a neural network to identify the source neuron, given an input window of interest In some cases, the vector-based back-end can directly execute the model. For high scales (e.g., 30,000 neurons), spike templates take up to 90% of the overall storage requirements. The template compression approach described herein reduces template storage by 8 to 11 times, while retaining greater than 99% relative accuracy to a high-performance spike sorter. Since the high-performance online spike sorter can be implemented in hardware, it provides power and area estimates for large-scale workloads. For example, it can sustain peak processing for 30 K neurons, consuming only 78.08mW (post-layout measurements scaled from 65nm to 7 nm).

For greater clarity, in the disclosure that follows, the following terms should be afforded the following meanings:

Probe: An invasive implantable device used to record electrophysiological signals from the brain; also known as neural probe.

Channel: A recording site of a probe; also referred to as an electrode.

Density: With respect to probes, density refers to the number of channels on a single probe. A higher density means a higher channel count.

Pitch: Distance between two adjacent channels.

Sample: A voltage reading from a channel at a given time.

Spike: The sequence of samples signalling a neuron's activation, typically 1-2 ms in duration.

Morphology: The shape of a spike which has particular characteristics.

Cluster: A group or set of N-dimensional points, often in the context of sorting or classification. An example is a collection of spikes.

Template: A proxy of a neuron's spike, identified by clustering. Typically, this is the centroid of a cluster.

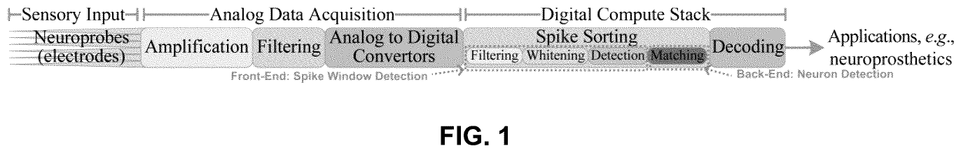

Generally, in a brain-machine-interface (BMI) system, neural signals must be collected, correctly attributed, interpreted, and then acted upon to induce a desirable effect. Generally, a BMI system includes a sensory input, analog data acquisition, and a digital computing stack composed of a spike sorter and an activity decoder. An example BMI pipeline is depicted in the diagram of FIG. 1.

Generally, the inputs to spike sorters are electrophysiological signals from neural probes. Neural probes are invasive implants that record, amplify and digitize voltages produced by neurons into streams. Modern probes have channel layouts which can vary from linear shanks, 2D grids, to 3D matrices. As the probes increase in density, the pitch can decrease to the micron range. Due to the proximity, spikes are often recorded on multiple nearby channels and provide spatial information. Various aspects of probe design influence the computations downstream, such as, the sampling rate, bitrate, number of channels, and layout. Sampling rates are commonly around 30 KHz and bitrates around 10-16 bits per sample. The number of channels generally ranges to upwards of tens of thousands and over time has shown exponential growth, necessitating improvements to software and hardware designs. Generally, the digital computing stack consists of 1) a spike sorter which aims to match each detected spike to the corresponding neuron that generated it, and 2) an activity decoder that deciphers the brain activity when reading groups of spikes.

Apart from the existing applications of spike sorting, including epilepsy detection and mitigation, treatment of Parkinson's disease, and cognitive control, larger scale applications remain unrealized due to the many stringent requirements to perform spike sorting at a high scale. Traditional spike sorters are generally not capable of keeping pace with the exponential growth in incoming data, requiring massively more computation and memory resources. Spike sorters have also seen drastic increases in algorithmic complexity, with further area and power constraints vital to advancements of untethered applications. The promise of such applications has been fueling a sustained wave of exponential growth in probe technology that continues unabated; probes containing thousands of electrodes (channels) are now common. At the same time, advances in the analog front-end have also kept up this rapid pace. However, present systems cannot meet the constraints for latency and portability in order to keep up with these advances.

Present approaches to spike sorters have significant technical limitations, for example: 1) they do not operate in real-time; 2) they are not accurate enough; 3) they are not portable due to being software solutions; and 4) for hardware solutions, they do not operate at the scale of modern probe technologies that require more efficient implementations for online spike sorting, especially if implantable BMIs are to be portable and responsive. While some spike sorters can handle up to thousands of neurons, such software-based spike sorters process offline after a recording has been stored; which, by its nature, severely limits the responsiveness and portability of the solution. For example, requiring a desktop-class GPU to achieve real-time performance, severely limiting portability.

For BMIs to effectively operate on thousands of neurons, the spike sorter must generally satisfy at least the following requirements: i) perform on-the-fly processing at real-time latency, ii) have low area and power energy costs for portability, and iii) scale to processing thousands of neurons very accurately. The processing must be done on-the-fly to be responsive, with a tight real-time latency budget (e.g., <50 ms for closed-loop manipulation). The area cost should also be considered, as desktop-class systems are not portable. Energy and power consumption must also be considered with untethered applications constrained to, for example, less than 2 W for portability and potential implantation. Alternative approaches to decode spikes without traditional spike sorters are generally limited on the number of electrodes and suffer from implementation issues, such as taking 10-20 seconds to process each electrode.

The goal of spike sorting is to discern when and which neuron “fires” given the raw output from the analog front-end. More formally, spike sorting is a source separation process which aims to attribute the recorded spikes to individual neurons, while separating background activity from local field potentials and noise (e.g. recording artifacts). This is challenging for several reasons: Firstly, while morphologically spikes look similar across neurons, their actual shapes vary in time, with the probe's placement, and by the neuron itself. Secondly, a channel can sense the superimposition of activity from many “nearby” neurons, as well as background activities in the brain. Thirdly, due to the lack of large in-vivo datasets, there is often no ground truth to appropriately determine accuracy. These factors jointly obfuscate the process, as it is difficult to discern whether similar spikes across nearby channels are from a single neuron or multiple. The challenges are addressed by, for example having an active research effort to improve spike sorting algorithms (and with it, a growing complexity), performance of neural experiments, culminating in the modern understanding of the foundational biophysics in the brain, and finally, developing synthetic datasets from the corpus of live cell models to provide ground truth data for objective and equal benchmarking.

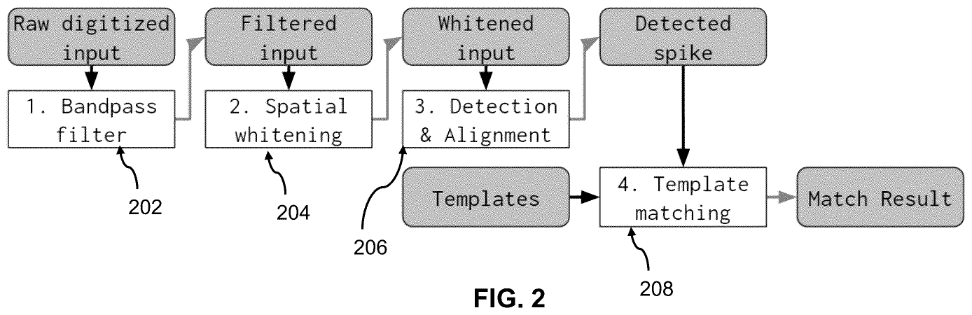

FIG. 2 is a diagram illustrating an online spike sorting pipeline, in accordance with an embodiment. In some cases, spike sorting can be performed offline after the full recording is available; however, in other cases, online processing can be used that utilizes spike templates that have been derived through prior offline runs. This approach is desirable for quick feedback and portability. Calibrating the templates offline can be used to tune to each subject and each application.

At block 202, bandpass filtering is performed. The incoming signals contain unwanted local field potentials at lower frequencies (e.g., <100-300 Hz) and high frequency noise (e.g., >3-6 kHz), both of which are filtered out. In a particular case, a 3rd order Butterworth filter can be used due to its widespread usage. In most cases, bandpass filtering can occur for every channel independently, scaling linearly with channel count.

At block 204, whitening is performed. After temporal noise is filtered, whitening removes spatially correlated noise from neurons that affect a large area, but are too far to be distinguished. Every channel has a whitening matrix derived from a covariance matrix of regions of silence. In some cases, local whitening can be performed where only nearby channels contribute to the covariance matrix, capping its total size to C×Cₜᵣ where Cₜᵣ<<C, and C the total channel count. Global whitening may be unnecessary due to negligible spatial noise from distant channels.

At block 206, detection and alignment are performed. The denoised activity is checked for spikes (i.e. if a neuron has fired). A particular approach uses an unsupervised threshold Thr=4 τ, where

$$σ = med \{ \frac{\vert x \vert}{0.6745} \}$$

and x is a long (e.g. 30 second) input stream for a channel. Thr is an estimate based on the median of the filtered signal which acts as a proxy for the standard deviation of noise. Signals crossing the threshold are classified as spikes. Other approaches can likewise be used, such as a nonlinear energy operator (NEO) detection approach or the use of deep neural networks. Downstream classification generally requires a window of samples centered at the trigger that is aligned at the peak amplitude. The spike's duration vary, but 2 ms can be held as a consensus (60 samples at a 30 kHz sampling rate). Spikes are often detected in a neighborhood of nearby channels with the maximum amplitude assigned as the central channel. A neighborhood provides spatiotemporal information (e.g., the relative amplitude and delay in sensing the trigger), improving classification accuracy. In an example, neighborhoods of the 9 closest channels can be used, and a maximum time difference of 10 samples between channels to account for intra-neighborhood delays.

At block 208, template matching is performed. To perform such matching, a vector dot-product of the input spike (9 channels by 60 samples) against pre-calibrated templates (same size as the spike) can produce a correlation score. The maximum correlation exceeding a threshold is detected as the source unit. The number of templates to compare against varies per channel. In an example, a maximum of 13 templates per channel, with an average of 3 and standard deviation of 2.3.

In some cases, the templates can be generated offline when calibrating the probe(s). Clustering divides the spikes into groups, where each group encloses spikes of a neuron. This is an unsupervised process where the number of neurons is not known beforehand. Generally, templates are the centroids of each cluster and approximate a neuron's spike. Templates are attributed to a single central channel where they have the strongest signal, while capturing the spike over the neighborhood of channels. This can change over time due to drift, although calibration of templates can resolve this. MountainSort4 can be used, for example, to generate templates.

TABLE 1 analytically models the memory footprint and computation costs of online spike-sorting (SS).

TABLE 1

| Unit | Stage | Insts./Second (B) |
|---|---|---|
| Front-End | Filtering | 25 |
| | Whitening | 70 |
| | Spike Detection | 0.08 |
| Back-End | Template Matching (F = 5) | 12.8 |
| | Template Matching (F = 20) | 50.8 |
| | Total (F = 5) | 107.88 |
| | Total (F = 20) | 145.88 |

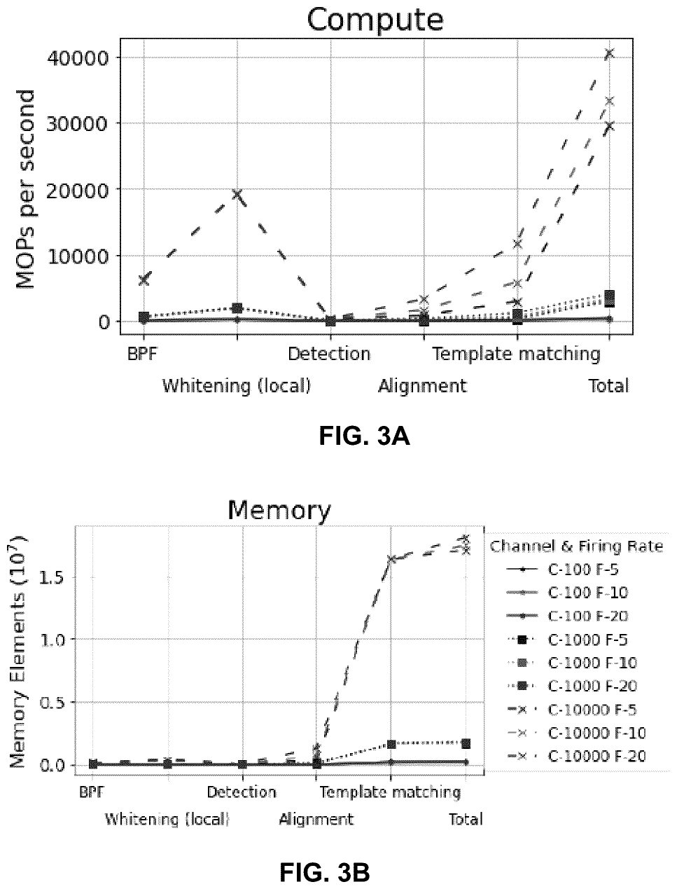

FIGS. 3A and 3B are charts showing how the above costs scale as a function of channel count C and firing rate F (in spikes/sec/neuron). Both parameters have nearly linear effects on costs. F has a minor effect compared to the dominant C. The computation and memory costs for C=100 are minimal, suggesting that even a software implementation with minor hardware assists may be sufficient and preferable for flexibility. For the present experiments, a high channel count scenario C=10 K neurons which fire at 5 Hz per neuron was considered.

In an example, to address the computational requirements, an optimized spike sorting pipeline in C, compiled for an i9-9900 K CPU using gcc with −O3 optimization and AVX extensions was developed. For a slow firing rate of 5 Hz and an average of 3 templates per channel, the program needed to execute

$$107 B \frac{instructions}{second} ,$$

which scaled up to

$$145 B \frac{instructions}{second}$$

for a 20 Hz firing rate. The CPU's power consumption of 95W alone makes it impractical for standard workloads. On the other hand, lower power processors were not able to handle the required instruction count for the pipeline. Modern GPUs are likely to meet the processing requirements but their power consumption is prohibitive. Example experiments indicate that an average of 57.4 W is required to maintain the desired firing rate, which exceeds general power constraints.

Embodiments of the present disclosure advantageously provide a significantly reduced power consumption, which can be less than 0.1 W when scaled. This highlights the potential for more power-efficient hardware solutions for SS pipelines with high neuron counts. In addition to the aforementioned challenges, the system is able to address the costs associated with memory requirements, particularly for template storage. With C=10 K and F=20, 16.2 million single-precision floating-point values (FP 32) are needed for template storage, making up 90.4% of the total memory costs.

Embodiments of the present disclosure provide template compression to extend the range of channel counts that can be practically processed in untethered applications. Templates are structured in three dimensions: scale, time and space. The scale is proportional to the number of neurons within the probe's detectable range, and grows linearly with channel count. Time is the number of samples in a template, proportional to a probe's sampling rate and spike width. Space is the neighborhood size.

In the past, manual datasets have been the primary source for assessing the performance of spike sorters. These datasets are often collected from juxtacellular recordings, where a probe is placed both internally for exact spiking information, and externally for validation data to mimic settings that are not privy to the internal data (as in practical applications). However, this is a very costly process in time and effort, requiring an expert to deftly insert an electrode into individual cells; an impractical approach for more than handfuls of data points. For decades, the neuroscience community has turned to synthetic generation of cell recordings for evaluation as a proxy with ground truth data.

In an example implementation, two separate datasets can be used to evaluate scalability. SpikeForest's (SF) datasets provide manual, synthetic and hybrid recordings, ranging from single-neuron and single-channel recordings up to 708 neurons and 64 channels. Recordings with a minimum of 10 neurons and 4 channels from 7 study sets composed of 29 studies or 87 recordings were used. To test for high scales, recordings were generated with a Neuropixel probe (NP) using a standard M E Arec flow. This NP dataset contains twenty 30-second recordings with 384 channels and 1500 neurons each. The NP datasets in three configurations were combined: 1500, 10,500 and 30,000 neurons or 1, 7 and 20 NP probes, respectively.

Templates are used as inputs to the compression approach and are matrices; for example, of size 60×9 (samples x channels around a center) FP 32 values. The samples per channel can be referred to as a waveform; in an example, 60 samples per channel to produce 9 waveforms to a template.

Generally, accuracy can be defined as the ratio of matching labels produced before (ground truth) and after compression (predictions). To quantify the memory costs and savings, a metric bits-per-value (BPV) can be used, which is agnostic to the size of the dataset and amortizes the memory cost.

$$B P V = \frac{Templatesbits + Metadataoverheadbits}{Numberoftemplatevalues}$$

A baseline can assume FP 32 values with no metadata overhead (BPV=32). Methods described herein aim to maximize compression (minimize BPV) while accounting for overheads.

Given the goals of portability and real-time performance, a lightweight low-energy decompression approach is extremely useful (compression is performed offline). The present embodiments can take advantage of at least three forms of similarity in neural signals: 1) similarities in relative dynamic ranges, 2) spatially across templates, 3) temporally within a template, along with other helpful optimizations to reduce the BPV.

To take advantage of similarities in relative dynamic ranges, quantization can be used to express values as fixed-point indices to a codebook. The value ranges can be further reduced by taking advantage of spatial similarity across templates, employing K-means clustering to find whole centroid waveforms (60 values in time from one channel). Waveforms of a cluster can be represented as per sample differences from their centroid. It is those differences that can be quantized into a codebook, as they have a significantly reduced dynamic range. Other quantization approaches can be used, each separating values into outliers and non-outliers. Outliers exceed a threshold magnitude and are stored in FP 32, whereas non-outliers are binned and replaced with a short index. This index points to the representative value stored in a codebook. Two example approaches are 1) Linear Quantization Enhanced (LQE) that evenly divides the value range into bins, using the mean of the values in each bin, and 2) Hierarchical Agglomerative Clustering (HAC) that evenly distributes values such that bins contain a similar number of values, giving more fidelity for high-density ranges.

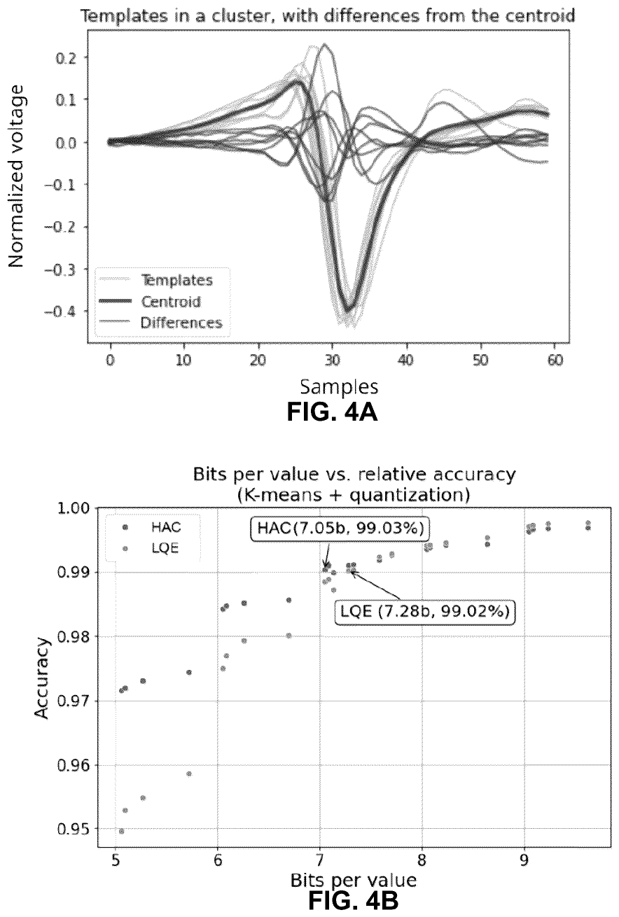

FIG. 4A is chart illustrating waveforms in a K-means clusters (the templates, the centroid, and their differences to the centroid). Note the reduction in the range of values for differences. FIG. 4B is chart illustrating an evaluation sweeping configurations of codebook sizes and outlier thresholds for K-means clustering and indirect quantization against their accuracies on the SpikeForest datasets.

The present embodiments take advantage of the temporal similarities within the spikes by encoding consecutive indices as deltas (Δ), where the first value is a base. Rather than using a fixed number of bits for all indices (e.g., 5 b for a 32 entry codebook), only as many bits as necessary are used (recorded in a metadata field) by removing any prefix of 0 bits. Delta encoding suffers when the spike transitions from/to the resting periods as the waveform exhibits abrupt changes in magnitude. The present embodiments provide Segmented Delta Encoding (SDE), which splits waveforms into multiple even segments, each encoded with its own base index. This is inspired by Base Delta Immediate (BDI) encoding, with two key differences. First, the bases themselves add no overhead as they are the first value of each segment (only the metadata to track the new length of Δ adds overhead; e.g. 3 b per segment to track lengths from [0, 7]).

Second, the delta encoding is fundamentally different, as it is calculated as consecutive differences rather than as a difference from a fixed base. This accounts for +30% greater compression on average than BDI. Under example multiple workloads, it was determined that 6 and 10 segments performed the best for LQE and HAC.

The exponents and mantissas can be trimmed to reduce overheads from overprovisioning by the FP 32 format. In an example, exponents can be losslessly trimmed to 2 and 4 bits for the outlier and centroid values, respectively, down from 8 bits while their mantissas can be lossily trimmed to 4 and 7 bits in order to retain an average accuracy above 99% (a negligible loss of 0.01%). Outliers are therefore reduced to 7 bits (1 sign, 2 exponent, 4 mantissa) and centroids to 12 bits (1 sign, 4 exponent, 7 mantissa).

Representing the original waveforms as shorter sequences of Δ often results in duplicates amongst these sequences. With a larger number of segments, the shorter sequences are more likely to be duplicates (only 11% at 10-segments are unique in the NP dataset, compared to 65% in the SF dataset). In some case, the duplicates can be encoded in a lookup table, and a 1-bit duplicate flag can be added, reusing the length and Δ fields in the templates as pointers to the lookup table. The pointer sizes and bit-lengths can be swept to find the optimal setting (9 b pointers for 2 b Δ).

TABLE 2 summarizes the reductions in BPV for each of the stages.

TABLE 2

Compression Method (Bits per value)

| | Template | | | |
|---|---|---|---|---|
| | Centroids & | | Datatype | Duplicate |
| Dataset | Quantize | SDE | Trimming | Dictionary |
| SF-HAC | 7.05 | 5.73 | 5.00 | 4.99 |
| SF-LQE | 7.28 | 4.78 | 3.94 | 3.93 |
| NP-HAC | 5.32 | 3.61 | 3.42 | 2.92 |
| NP-LQE | 5.64 | 3.33 | 3.06 | 2.83 |

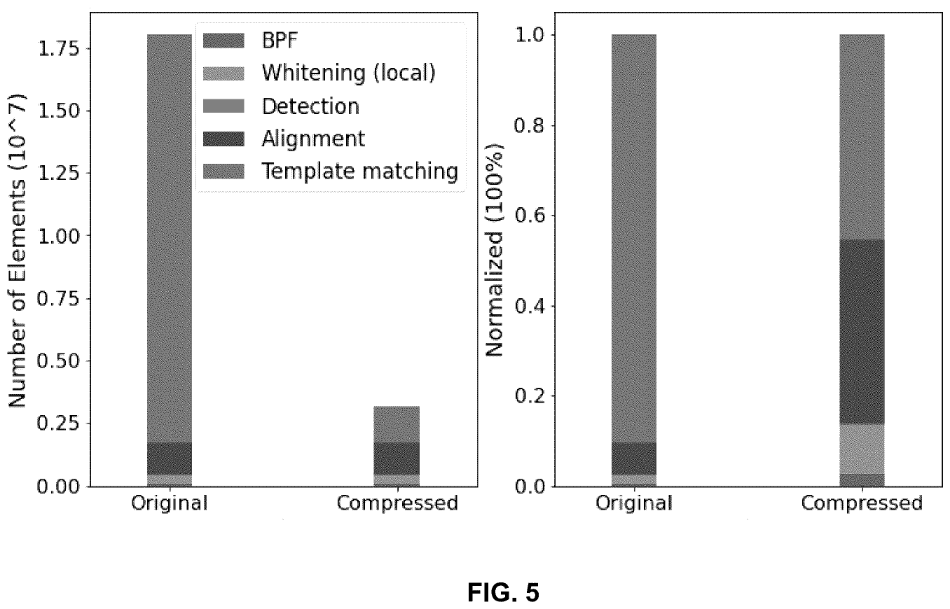

FIG. 5 illustrates the effect of template compression on overall memory footprint. The present approaches provide compression methods that reduce overall memory footprint to 1.4 MB, a 5.7× reduction over the baseline. The memory cost of an online spike sorter for 10 K channels and 20 Hz firing rate from our analytical model, (Left) the absolute difference, (Right) the normalized memory costs.

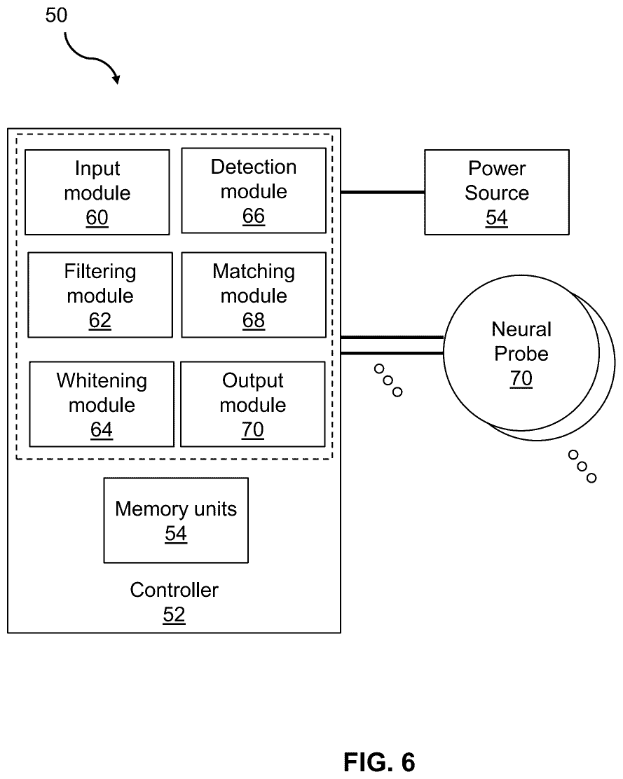

Turning to FIG. 6, shown is a conceptual diagram of a system 50 for low-memory parsing of electrophysiological signals from neurons to identify neuron activity, according to an embodiment. The system 50 comprises a controller 52, a power source 54 (e.g., batteries), and a plurality of neural probes 70. The neural probes 70 are electrically connected to the controller 52. The neural probes 70 can be, for example, quad-shank Michigan probes, NeuroSeeker probes, Neuropixel probes, Utah arrays, a 3D NeuroNexus matrix, or the like. In some cases, the controller 52 can include a field-programmable gate array (FPGA), one or more processors, or other circuitry. In some cases, the controller 52 can include or be in communication with one or more memory units 54, such as flash memory or static random-access memory (SRAM). In further cases, the controller 52 can be implemented using dedicated circuitry. The controller 52 can also include an interface to interface with other devices/elements or other computing or networking devices. The system 50 can include circuitry for power management located associated with the power source 54 or the controller 52.

The controller 52 can execute a number of conceptual functional modules either in hardware or software, as appropriate; for example, executing the functions of the modules from instructions received from the memory units 54 on one or more central processing units, one or more graphical processing units, microprocessors, dedicated hardware, or other integrated processing circuits. Such conceptual modules can include an input module 60, a filtering module 62, a whitening module 64, a detection module 66, a matching module 68, and an output module 70. The functions of the modules can be combined or performed on further conceptual modules.

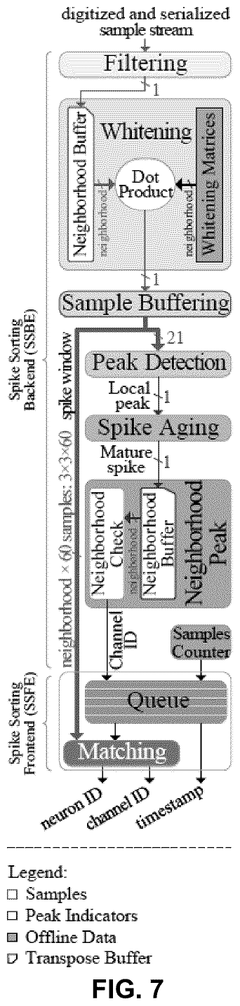

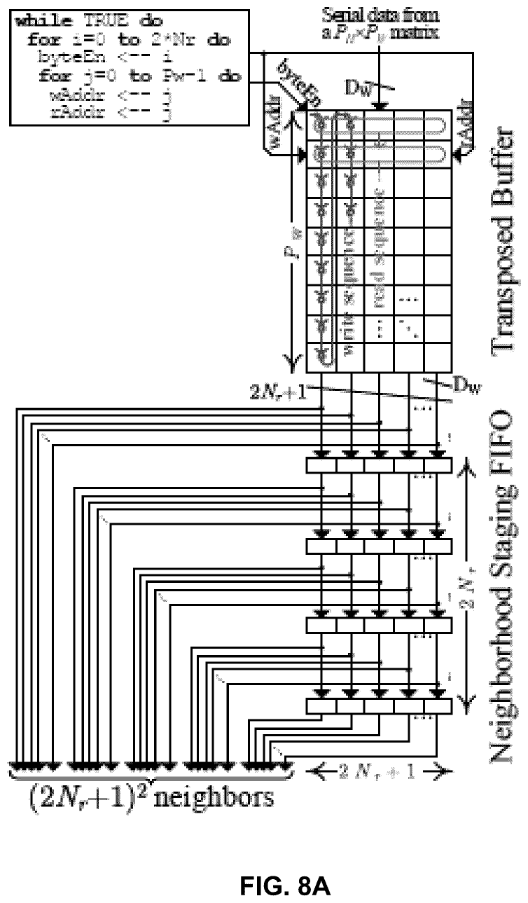

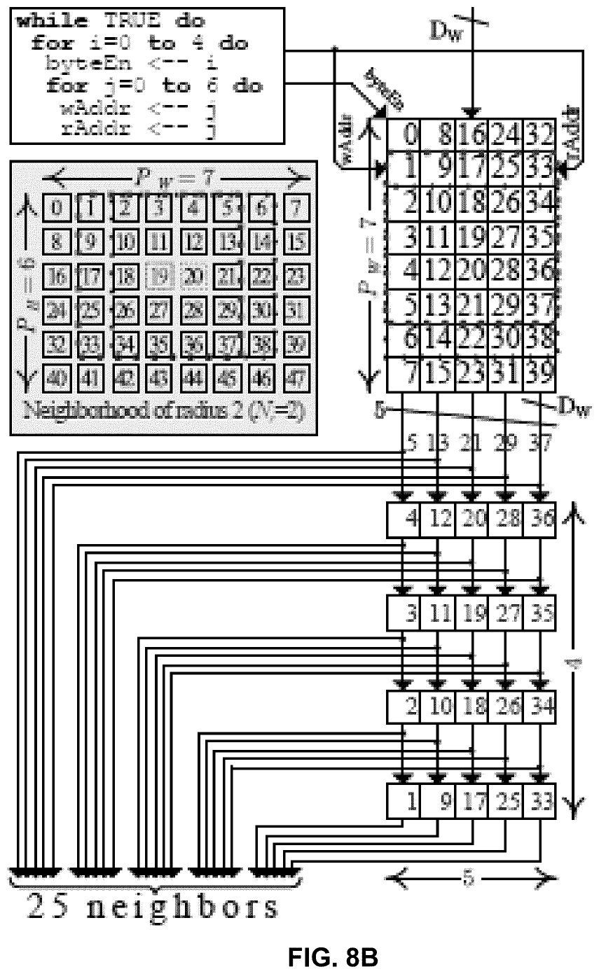

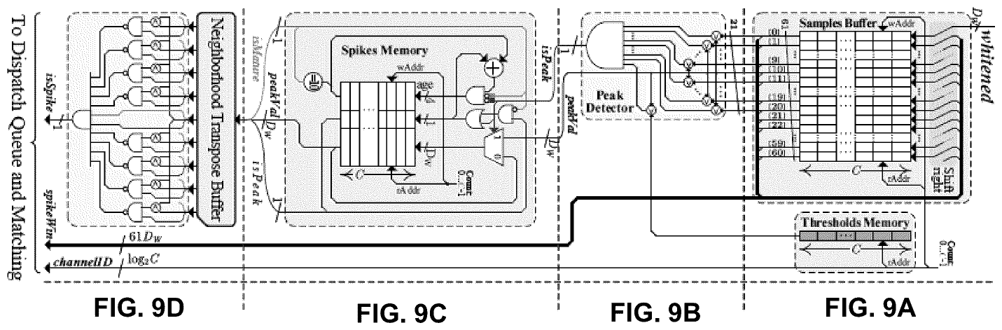

FIG. 7 is a diagram of an example pipeline for spike sorting, in accordance with the system 50. Data can flow between stages either directly or via scratchpad memories. The organization can be optimized by utilizing the input sample flow from the analog front end. FIGS. 8A and 8B illustrate the digitization process: data is converted into Q-bit (up to 16-bit) format using ADCs and then serialized across C channels. Consequently, the spike sorting pipeline processes a single 16-bit sample per cycle. For a standard sampling rate of fₛ=30 kHz and a desired channel count of C=10 K, achieving real-time feedback necessitates an operational frequency of fₒₚ=300 MHz. FIG. 8A shows a neighborhood transpose buffer and FIG. 8B shows an example with P_{W}=7, P_{H}=6, Nᵣ=2, neighborhood center at channel 19 and 20 . FIGS. 9A to 9D show four stages of spike sorting, where FIG. 9A shows a sample buffering stage, FIG. 9B shows a peak detection stage, FIG. 9C shows a spike aging stage, and FIG. 9D shows a neighbourhood peak stage.

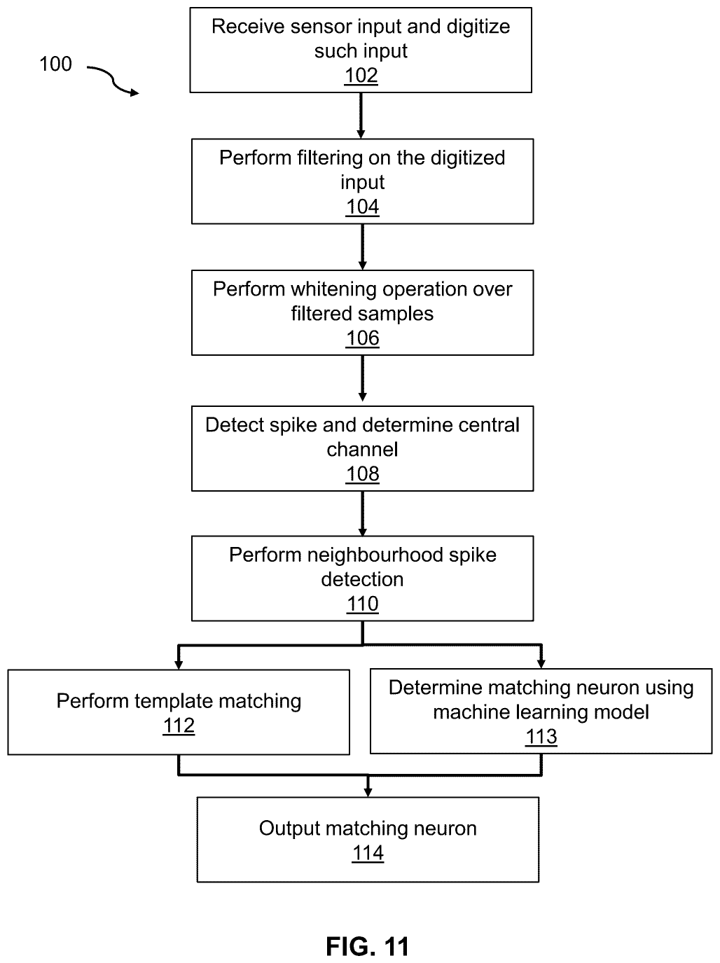

FIG. 11 illustrates a method 100 for low-memory parsing of electrophysiological signals from neurons to identify neuron activity, in accordance with an embodiment. Generally speaking, the method performs digital filtering over the samples of a single channel. The filtered samples go through a Neighborhood Buffer which enables Whitening to seamlessly operate on samples from a neighborhood of channels. From this, identification of where (i.e., which channel) and when spikes occur can be determined, and a window can be provided (for example, a window of 60 samples per channel over a neighborhood of 3×3 channels) to template matching, or to a machine learning model, for identification of the source neuron.

At block 102, the input module 60 receives electrophysiological input signals from the one or more neural probes and digitizes, and in some cases serializes, such received input signals.

At block 104, the filtering module 62 performs filtering on the digitized input. In an example case, a 3rd order Butterworth infinite input response (IIR) bandpass filter with a cascaded biquads can be implemented. For each FP 32 output, the filtering module 62 can perform 12 multiplications plus 11 additions over what is effectively a 6 sample window. Given the relatively low sampling rate (30 kHz), time-domain multiplexing multiple channels to a filter reduces costs. A single set of multipliers and adders is sufficient as the system 50 can time-multiplex them over the channels via a 10 K scratchpad (one row per channel). Each row contains 6 FP 32 values enabling a 6-stage pipelined filter implementation. Each cycle, the filtering module 62 can read one row and write another. The scratchpad can be banked and since the filtering module 62 processes channels round-robin, each bank is single ported. Every cycle, the filtering step can produce a single FP 32 sample.

At block 106, the whitening module 64 can perform a whitening operation over the filtered samples of a group of channels. The channels of a probe can be arranged in a uniform grid, which can be denoted as P_{W}×P_{H}. From a central channel, its neighbors are channels within a distance of Nᵣ (neighborhood radius). FIG. 8A shows an example of a 7×6 channel probe and a neighborhood centered at channel 19 with a radius of Nᵣ=2. In an example, whitening samples from channel 19 needs samples from the whole neighborhood in the same time frame. If the incoming data is organized line-by-line in memory, multiple read ports may be required as reading a whole neighborhood requires buffering until all channels are read. Instead, the whitening module 64 can use a neighborhood buffer (NB) to minimize buffering, use single-ported memories, and perform one write and read access per cycle while maintaining throughput. As FIG. 8A shows, the NB comprises the Transpose Buffer and the neighborhood FIFO. wAddr and rAddr denote the writing and reading addresses of the transposed buffer, respectively. byteEn selects a column in line wAddr of the transposed buffer for writing, while all other columns stay unchanged. The incoming data (one value per cycle) is written into the transpose buffer column-wise. The samples from a row of channels in the probe are organized as a column. The width of the transpose buffer is 2 Nᵣ+1, the same as the width of a neighborhood, while its depth equals 8 to the width of the probe matrix, P_{W}. In an example, since a target neural probe has C=10 K channels, P_{W} may reach 100 for a square-shaped probe. Once the last element of the neighborhood is written to the transpose buffer, the whole neighborhood is in the latest 2 Nᵣ+1 lines. These lines can be read sequentially, just ahead enough, and pushed into the neighborhood staging FIFO. For example, in FIG. 8B, the row containing 4-36 is read out from the transpose buffer when 37 is written into it, whereas the line containing 5-37 is read out when writing 38. At that point, the neighborhood staging FIFO contains the neighborhood for 19 which can be whitened. A transpose buffer can be implemented, for example, as several single-ported SRAM banks. Each cycle, a single filtered value is written to one bank and a line is read from another.

A channel i's whitened value is the dot-product of all its neighbors and a precomputed whitening matrix, whitened(i)=neighbors(i)·whiteningMatrix(i). The whitening module 64 receives, generally en masse, the channel-wise serialized, filtered data from the neighborhood FIFO via dedicated connections. The whitening module 64 then reads the corresponding whitening matrix and performs the dot-product. The per channel whitening matrices can be stored, for example, in a C row SRAM, where row i contains the (2 Nᵣ+1)² whitening coefficients for the neighborhood around channel i. Each neighborhood can contain 9 values and the whitening matrices table can thus contain 10 K×9 FP 32 coefficients. Whitening generally produces one FP 32 value per cycle.

Prior to performing template matching, at block 108, the detection module 66 needs to detect that a channel has a spike, and determine the central channel, as spikes may be picked up by several neighboring electrodes. The samples from the neighborhood can then be used for template matching. Specifically, once the detection module 66 determines that a spike occurred in channel c and a time t, template matching will need the samples from multiple channels. For example, the central channel and from the eight neighboring channels that surround it; such as channels 19 and 10-12, 18, 20, and 26-28 respectively in FIG. 8B. From each of those channels, the system 50 requires a number of samples around t; for example, 60 samples around t being 20 before and 39 after. In a first stage, a spike can manifestas a peak, which the detection module 66 first detects locally within a channel. In an example, peaks occur when a sample is larger than ±10 samples in time. This detection is done for all channels in “Sample Buffering” and “Peak Detection” stages. This stage can also buffer the full window that template matching needs once the central channel is identified. In a second stage, a detected peak is determined to be a true peak if none of its neighbors has a higher peak within, for example, 10 time steps. This condition can be checked by, first, ensuring that the peak “matures” (e.g., stays in the buffer for 10 samples before checking with its neighbors, which is referred to as “Spike Aging”), and second, checking if the spike is the highest amongst its neighbors within a given number of samples (e.g., within ±10 samples, referred to as “Neighborhood Peak”).

A spike is pivoted by a centered peak detected by a sample that exceeds a per channel threshold and is greater than, for example, ±10 neighboring samples in time. Once the peak is detected, a full spike window is passed for template matching. In an example, the full spike window can include the 60 samples around the detected spike. This functionality can be implemented by the detection module 66 by buffering the last number of samples per channel (e.g., 60) in a Sample Buffer (SB), as illustrated in FIG. 9A. The SB contains C rows, one per channel. The whitened values are written in the first column of the SB one at a time. In steady state, a full row (e.g., 60 samples) is read out each cycle, shifted right to include the new incoming whitened sample, and written back to the buffer (a cycle later to allow single ported memories). The Peak Detector determines whether a peak has occurred in, for example, the 21 most recent samples by comparing the 11th sample (center) with the 10 before and after it and with a per channel threshold. If a peak is detected, the channel number (ChannelID), peak indicator (isPeak), and the peak value (peakVal) proceed to the spike aging counter (SAC) stage, which aids with neighborhood peak detection. The SAC ensures that during the next given number of timesteps (e.g., 10 timesteps), the magnitude of this local peak is compared against any other locally detected peaks in the neighboring channels. If this peak happens to be on the central channel, then an entry is pushed in the matching stages on a first-in-first-out (FIFO) basis. At that point, the peak will be in a desired position (e.g., position 20 as the row has shifted by 10 positions). Template matching occurs a given number of timesteps later (e.g., 30 timesteps later) when the peak will be appropriately centered for peak detection, reading the corresponding samples directly from the SB.

Once a spike is detected, at block 110, the detection module 66 can perform neighbourhood spike detection by checking that none of its neighbors also have spikes within a given number of samples timeframe (e.g., within a 10 sample timeframe). To perform this check, the age of each spike can be stored and maintained in a Spikes Memory as shown in FIG. 9C. Each row in the Spikes Memory includes three fields, the relative age of the spike (e.g., in samples, 0-10), a single bit indicating a peak, and the peak value. There is one row per channel.

If an input peak is detected (isPeak is asserted), the age field will be zeroed, the peak indicator will be set high, and the peak value (peakVal) in the input will be stored into the memory line corresponding to the same channel. In the subsequent samples of the same channel, the age will be increased until it reaches maturity, for example, 10 sampling cycles. When a peak entry matures, its three fields (maturity indicator isMature, isPeak, and peakVal) can be written into the transpose buffer of the neighborhood peak detection stage.

The purpose of neighborhood peak detection is to check for each spiking channel that no neighboring channel also has spike with a larger magnitude within a given timeframe (e.g., within a 10 samples timeframe). FIG. 9D shows that this stage is composed of two elements, a transpose buffer accepting entries from the aging unit, and a neighborhood check that performs the neighborhood check. Since the neighborhood is, for example, 3×3, the transpose buffer is organized as P_{W} rows (e.g., 3 entries each). Each entry contains a peak value, and peak and maturity indicators. Using a similar access strategy to the NB, the exemplary 3×3 entries are read into the output FIFO, where the check occurs for the entry in the center. If the test succeeds, the spike indicator isS pike is asserted, and an entry is placed in the Dispatch queue and tagged with a 40 bit counter for identification. Once the full sample window (spikeWin) has entered the SB (for example, delaying 10 more timesteps to center the window at the peak), the samples can be copied and template matching can be performed.

At block 112, the matching module 68 performs template matching in order to match a neuron for the detected spike as an identification of such neuron activity. The template matching accepts a window of samples per channel from a neighbourhood of channels (spikeWin); for example, 60 samples per channel from a 3×3 neighborhood of channels. The samples are copied from the SB using the ChannelID from the dispatch queue. The dispatch queue contains αC entries, where 0<a<<1 as spikes occur relatively infrequently. In an example, setting α=0.04 resulted in no stalls in the example experiments. Template matching can be performed by determining a dot product of the neighbourhood of samples (e.g., 3×3×60 samples) from the Samples Buffer with one or more templates. The center channel index can be used to fetch the templates. The matching neuron corresponds to the highest magnitude dot product. This approach can be implemented as a vector datapath comprising several multiply accumulate units. For example, a 16-wide datapath ensures that the matching stage can process incoming spikes at the exceedingly rare peak rate of 20 Hz per neuron and 13 templates per channel.

The templates can be stored as a fixed portion and a variable portion, decompressing on demand using the ChannelID. If, for example, every template contains 60 samples per channel (referred to as a waveform) across 9 channels, the fixed storage consists of a 4 b centroid tag, and six 9 b metadata chunks (5 b bases+1 b DF+3 b lengths); for a total of 58 b per waveform. A row of fixed memory can then be stored as nine 58 b segments (by 30,000 columns, the number of templates) accessed with 15 b indices. The number of templates for each channel can be inferred as the difference between the current and next index, which is commonly 3 but can be as much as 13. The variable storage consists of a variable length Δ. In this example, a 9×9 grid of memory blocks (Δ×neighbors) can be used for storage. Δ are packed in 9 virtual columns in segment order allowing efficient expansion into 5 b. Having 9×9 memory blocks enables parallel access to each of the 9 values of a segment, and each of the 9 waveforms. Since the 9 values of a segment have the same bitwidth, the system 50 can load all segments of a template in 6 cycles.

To fully decompress a single waveform, the centroid tag can be used to extract a centroid waveform, e.g., for a 4 b centroid tag, sixty 12 b values from the 16-row centroid table. In parallel, the segments can be loaded as above. The base can be forwarded to a Δ decoder. In an example, for DF=0, the length corresponds to the size of each of nine Δ ([0,5 b]). For DF=1, the Δ are treated as a 9 b pointer to a 512-entry table with nine 2 b Δ which are the segment. Each Δ is consecutively added to the base to reproduce the index to a 32-entry quantization codebook. The codebook value is added to the corresponding centroid value to reproduce the original template value. Parallel access can be used for the Δ decoder and codebook for acceleration. Outliers can act as an override for the decompressed value from the codebook, as outliers typically must still be added to the centroid. In an example, a maximum of 1.1% of all values (178 k) were classified as outliers; however, more outliers can be provisioned for, such as up to 200,000 outliers. When loading templates, the system 50 locates the number of waveforms that contain outliers. This can be inferred by reading two consecutive entries of an outlier pointer memory for the starting count and the subsequent count of waveforms with outliers for the current template, and indexes into the offsets needed to locate the position and outlier value. Since, generally, only 10% of waveforms have outliers, in an example, the index only needs 27,000 entries of 4 b for the segment and 19 b for the offset into the larger 200,000 memories. In this example, the outlier position is 6 b (for its position in the template), and the value itself is 7 b.

At block 114, the output module 70 outputs the matching neuron as an identification of such neuron activity, where the output can be communicated to other systems/devices or saved in memory.

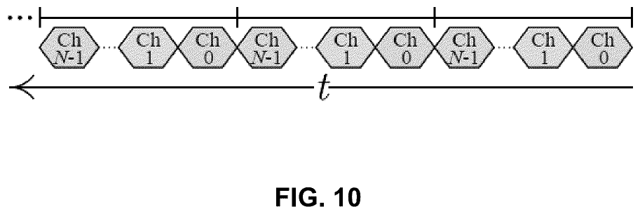

FIG. 10 illustrates an example schedule of incoming data. Samples from each channel are digitized and serialized. The outputs of the filtering and the whitening stages can follow the same schedule.

The present inventors conducted example experiments to evaluate the performance of the system 50. In such experiments, a configuration with 10,000 channels was implemented using a commercial 65 nm process. This configuration is capable of supporting low latency BMIs, which require a sampling rate of 30 KHz. The target operation frequency was set to 300 MHz for achieving optimal performance. The units were implemented in Verilog and synthesize with the Synopsys Design Compiler. Layout used Cadence Encounter and Synopsys' commercial Building Block IP library. Power was estimated via Encounter. Nominal operating conditions were used to model power and latency. SRAM buffers were modeled using CACTI. TABLE 3 summarizes the post-layout logic and memory costs for each of the modules to quantify the area and power consumption. Both area and power are dominated by the memories in Sample Buffering+Peak Detection and Template Matching+Decompression, accounting for 18% and 77% of total area and 44% and 33% of power, respectively. However, much of the power costs are due to standby leakage (598 mW, 45%). We estimate the system's 50 power use and area with more recent technologies using the methodology of Stillmaker and Baas. TABLE 4 shows total power and area estimates with technology nodes varying up to 7 nm. Scaling the system 50 to 7 nm would reduce the area and power to 4.25 mm² and 78.94 mW, respectively. Due to the specificity and constraints of a portable online spike sorter, the system 50 should use fine-grained customization for accurate implementation.

TABLE 3

| | Area | Area | Area | Power | Power | Power |
|---|---|---|---|---|---|---|
| | (Logic) | (Memory) | (Total) | (Logic) | (Memory) | (Total) |
| STAGE | mm² | mm² | mm² | mW | mW | mW |
| Filtering | 0.122 | 1.532 | 1.654 | 35.22 | 46.36 | 81.58 |
| Whitening (Pipelined) | 0.225 | 2.926 | 3.151 | 62.72 | 69.04 | 131.76 |
| Sample Buffering + Peak Detection | 0.034 | 19.436 | 19.471 | 7.48 | 576.25 | 583.73 |
| Spike Aging + Neighborhood Peak | 0.005 | 0.366 | 0.371 | 0.63 | 5.51 | 6.14 |
| Dispatch Queue | 0.028 | 0.04 | 0.068 | 12.86 | 0.23 | 13.09 |
| Template Matching + Decompression | 0.447 | 83.869 | 84.316 | 171.00 | 437.21 | 608.21 |
| Total | 0.861 | 108.169 | 109.03 | 289.9 | 1134.6 | 1424.51 |

TABLE 4

| | | |
|---|---|---|
| Tech. Node | Power (mW) | Total Area (mm²) |

| | | | |
|---|---|---|---|
| 65 | nm | 1424.51 | 109.03 |
| 45 | nm | 881.28 | 71.96 |
| 32 | nm | 443.93 | 33.8 |
| 20 | nm | 256.02 | 15.26 |
| 16 | nm | 172.38 | 14.17 |
| 14 | nm | 133.39 | 13.08 |
| 10 | nm | 107.1 | 7.41 |
| 7 | nm | 78.94 | 4.25 |

In a further embodiment, instead of template matching at block 112, the matching module 68 can, at block 113, use a machine learning model, such as a Convolutional Neural Network (CNN), to determine the matching neuron for identification of neuron activity. The CNN accepts the same input as template matching, and the ChannelID, outputting a vector for the firing neurons ( [symbol] ). TABLE 5 details an example architecture for such model (where applicable the stride is 2), 3 configurations evaluated, and the compute and memory costs during inference. Hyperparameters for model size and training were empirically derived. Training times ranged from 2-12 hours on a NVIDIA Geforce RTX 3090 GPU. Performance was measured on the NP datasets as the 5-fold cross validation accuracy. Template matching's accuracy for these extremely large datasets was 67%, whereas the small, middle, and large CNNs achieve accuracies of 85.6%, 89.9% and 91.9%, respectively. However, practical deployment of CNNs are difficult, memory demands of even the small models exceed template matching for 30,000 neurons. The 1,000 neuron configuration with the small model could be practical for simple applications as it requires 1.48 GOPs and about 1.6 MB of storage. However, with 30,000 neurons (10,000 channels) the demands may exceed 1.48 TOPs even with the lower F=5.

TABLE 5
Model Configurations

| Parameter | Small | Medium | Large |
|---|---|---|---|
| N | 16 | 32 | 64 |
| I | 513 | 1025 | 2049 |
| J | 256 | 512 | 1024 |
| k | 128 | 256 | 512 |

Model Architecture

| Layer | Type | Dimensions |
|---|---|---|
| 1 | 1D Conv | n × 58 |
| 2 | Max Pool | n × 29 |
| 3 | Squeeze Exc. | n × 29 |
| 4 | 1D Conv | 2 × n × 29 |
| 5 | Max Pool | 2 × n × 14 |
| 6 | Adapt. Avg. Pool | 1 × i + channelID |
| 7 | Fully Conn. | 1 × j |
| 8 | Fully Conn. | 1 × k |
| 9 | Fully Conn. | 1 × N |

In contrast to other software approaches, the present embodiments do not have to be run primarily on desktop CP Us/GPUs or server class hardware, avoiding the necessity to incur large energy costs and reduced portability. Additionally, in contrast to other hardware approaches, the present embodiments do not have to sacrifice accuracy and scalability for the sake of implementation and form factors. For high-density probes, this sacrifice is problematic because: 1) neurons are often detected on multiple probes (one neuron is picked up by many probes), and 2) having several probes in close proximity allows the system 50 to discern among multiple neuron groups that are nearby (many neurons are picked up by several probes in a way that allows us to discern which one it was). Therefore, in contrast to other approaches, the system 50 will not detect each spike multiple times, once per neighboring channel, and will be able to discern among multiple neurons that are detected from the same electrode. Other hardware approaches merely perform a subset of stages, such as spike detection but not spike sorting, and thus, target different types of applications. Overall, other hardware approaches only handle input from few channels, limiting real-world applications where coarse-grain neural decoding is sufficient and, therefore, allows for use of only very simplistic spike sorting. In contrast, advantageously, the present embodiments effectively handle a large number of channel inputs, can use advanced spike sorting approaches, and can cover a wide variety of real-worlds applications; thus, significantly assisting to increase the development of impactful BMIs.

Scalability is a pressing problem with modern neural recording devices, requiring innovative solutions to keep pace. To address such problems, the present embodiments provide a lightweight template compression approach and an accelerator for performing the computations in real-time. Additionally, since the system's 50 components are modularly designed, the constituents can be independently optimized for specific constraints.

Although the foregoing has been described with reference to certain specific embodiments, various modifications thereto will be apparent to those skilled in the art without departing from the spirit and scope of the invention as outlined in the appended claims.

1. A method for low-memory parsing of electrophysiological signals from neurons to identify neuron activity, the method comprising:
receiving electrophysiological signals from neural probes;
digitizing the received electrophysiological signals and serializing the digitized signals across a plurality of channels;
performing filtering on the digitized electrophysiological signals of each channel;
performing whitening over the filtered samples of a group of associated channels;
detecting whether the whitened samples for each channel comprises a spike, the samples comprise the spike where a centered peak exceeds a threshold and is greater in value than a predetermined number of neighboring samples;
determining a matching neuron for the detected spike as an identification of neuron activity; and
outputting the identification of neuron activity.

2. The method of claim 1, wherein filtering is performed using a third order Butterworth infinite impulse response bandpass filter with a cascaded biquads.

3. The method of claim 1, wherein performing filtering comprises performing time-domain multiplexing with the digitized electrophysiological signals of multiple channels.

4. The method of claim 1, wherein the group of associated channels are arranged in a uniform grid for whitening.

5. The method of claim 1, wherein performing whitening comprises determining, for each one of the group of associated channels, a dot product of neighboring samples of the channel and a predetermined whitening matrix.

6. The method of claim 1, wherein detecting whether the whitened samples for each channel comprises a spike comprises determining a central channel of the group of associated channels that has the spike.

7. The method of claim 1, wherein determining the matching neuron comprises determining a dot product of the neighboring samples with one or more templates, the matching neuron corresponding to a highest magnitude dot product

8. The method of claim 7, wherein templates for template matching are each stored as a fixed portion and a variable portion which is decompressible.

9. The method of claim 8, wherein performing template matching comprises decompressing the variable portion of each of the templates, wherein decompressing the variable portion of each template comprises overriding a decompressed value with an outlier.

10. The method of claim 1, wherein determining the matching neuron comprises using a trained machine learning model to determine the matching neuron for identification of neuron activity.

11. A controller for low-memory parsing of electrophysiological signals from neurons to identify neuron activity, the controller comprising hardware to receive instructions from one or more memory units to execute:
an input module to receive electrophysiological signals from one or more neural probes that capture the electrophysiological signals, to digitize the received electrophysiological signals, and to serialize the digitized signals across a plurality of channels;
a filtering module to perform filtering on the digitized electrophysiological signals of each channel;
a whitening module to perform whitening over the filtered samples of a group of associated channels;
a detection module to detect whether the whitened samples for each channel comprises a spike, the samples comprise the spike where a centered peak exceeds a threshold and is greater in value than a predetermined number of neighboring samples;
a matching module to determine a matching neuron for the detected spike as an identification of neuron activity; and
an output module to output the identification of neuron activity.

12. The controller of claim 11, wherein the whitening module comprises a neighborhood buffer to receive the filtered samples of the group of associated channels, the neighborhood buffer comprising a transpose buffer that feeds a neighborhood staging.

13. The controller of claim 11, wherein the detection module comprises a sample buffer to receive a last number of samples per channel, and a spike aging counter to perform peak detection for the predetermined number of neighboring samples.

14. The controller of claim 11, wherein filtering is performed by the filtering module using a third order Butterworth infinite impulse response bandpass filter with a cascaded biquads.

15. The controller of claim 11, wherein filtering is performed by the filtering module by performing time-domain multiplexing with the digitized electrophysiological signals of multiple channels.

16. The controller of claim 11, wherein the group of associated channels are arranged in a uniform grid for whitening.

17. The controller of claim 11, wherein the whitening module performs whitening by determining, for each one of the group of associated channels, a dot product of neighboring samples of the channel and a predetermined whitening matrix.

18. The controller of claim 11, wherein the detection module detects whether the whitened samples for each channel comprise a spike by determining a central channel of the group of associated channels that has the spike.

19. The controller of claim 11, wherein the matching module determines the matching neuron by determining a dot product of the neighboring samples with one or more templates or comprises using a trained machine learning model to determine the matching neuron for identification of neuron activity.

20. A system for low-memory parsing of electrophysiological signals from neurons to identify neuron activity, the system comprising the controller of claim 11, a power source connected to the controller, and the one or more neural probes electrically connected to the controller.
